(19) United States
(12) Patent Application Publication
(10) Pub. No.: US 2025/0017873 A1
(43) Pub. Date: Jan. 16, 2025

(54) TOPICAL PEEL-OFF CREAM FOR NUMBING PAIN DURING SKIN-RELATED PROCEDURES

(71) Applicant: Synersia, Inc., Charlottesville, VA (US)

(72) Inventors: Haeley Wotnosky, Wake Forest, NC (US); Rithika Anand, Centreville, VA (US)

(21) Appl. No.: 18/773,435

(22) Filed: Jul. 15, 2024

Related U.S. Application Data
(60) Provisional application No. 63/527,007, filed on Jul. 15, 2023.

(51) Int. Cl. A61K 31/045; A61K 45/06; A61K 47/10; A61K 47/12; A61K 47/18; A61K 47/32; A61K 47/44

(52) U.S. Cl. CPC A61K 31/045; A61K 9/0014; A61K 9/06; A61K 31/167; A61K 31/245; A61K 45/06; A61K 47/10; A61K 47/18; A61K 47/32; A61K 47/44

(57) ABSTRACT

A topical cream for numbing pain during skin-related procedures.

### CROSS-REFERENCE TO RELATED APPLICATION

The present application relies on the disclosures of and claims priority to and the benefit of the filing date of the following U.S. Provisional Patent Application:
U.S. Appl. No. 63/527,007, filed Jul. 15, 2023.

The disclosures of that application are hereby incorporated by reference herein in their entireties.

### FIELD OF THE INVENTION

In various embodiments, the present invention relates to topical creams for numbing pain during dermatological or other skin-related procedures, including but not limited to tattooing procedures, injections/phlebotomy, and laser procedures.

### BACKGROUND

Procedures involving the skin can be painful and uncomfortable. Topical creams have been developed to numb the skin and reduce pain during these procedures. However, the currently available topical numbing creams have several drawbacks.

For example, most numbing creams on the market take a significant amount of time to dry or never dry at all. Others cause skin irritation or allergic reactions, making them unsuitable for certain patients or inviting additional complications to the procedure. Other problems with existing creams include the amount of time it takes to remove them, failure to numb effectively, lack of FDA approval, distribution channels through compounding pharmacies (which add expense, create difficulty for patients and clinicians to pick up the cream, and sometimes requires the patient to apply the cream at home prior to their procedure, which creates toxicity risks), and the need to occlude the cream with plastic wrap or another tool.

Therefore, there is a need for an improved composition of a topical cream that can numb pain quickly, provide long-lasting pain relief, and is well-tolerated by patients.

### SUMMARY OF THE INVENTION

The present invention provides an improved composition of a topical cream for numbing pain during dermatology procedures. The combination of lidocaine, prilocaine, tetracaine, and/or benzyl alcohol, along with the use of one or more of a codelivery agent, a skin-protective agent, an occluding agent, and/or a peel-off agent, make embodiments of this cream unique in comparison to currently available topical numbing creams on the market. In embodiments, the clinical composition contains lidocaine and tetracaine for use by a clinical provider in a clinical setting, and another embodiment of the cream comprises lidocaine and/or prilocaine for use over-the-counter (“OTC”). The cream may also include, in aspects, the use of additional enhancing agents, such as antimicrobial agents and wound healing agents to support the healing of injuries, including but not limited to burns, cuts, and blisters. The cream may also act as a liquid bandage to protect injured skin. In embodiments, a single agent can act in multiple roles (e.g., a single agent can act as a codelivery agent and an occlusive agent).

Compared to currently available topical numbing creams, the present invention is easier to procure, apply, and remove. At its clinical strength, the present invention may contain the highest concentration of numbing agents in accordance with regulatory guidelines and could be purchased in advance and stored by clinicians, without the need to order and pick up a product from a compounding pharmacy. The present invention also dries relatively quickly, reducing inconveniences to the patient (especially when numbing the lips, during which time the numbing cream often leaks into the patient's mouth), and allowing the patient to move about freely during the waiting period. In embodiments, the present invention self-occludes, which eliminates the need to wrap the cream with any additional materials and increases the penetration of active particles into the skin for a stronger numbing effect. The present invention can be made with ingredients which have minimized side effect profiles, which aims to reduce reports of skin irritation or allergic reactions. Furthermore, the present invention, in aspects, peels off.

Additionally, in embodiments, the creams disclosed herein include a higher percentage of numbing agents to increase the numbing effect as compared to existing agents. The cream, in aspects, can also include a self-occlusive property in addition to a peel-off property to trap the numbing agents and send them deeper into the skin for an enhanced numbing effect as compared to existing creams. Absorption of the product into the skin can be accomplished through the codelivery of active numbing agents with a codelivery agent, in aspects of the current invention. The cream of the current invention can also interact with the top layer of the skin by creating biochemical changes and mechanical disruption (e.g., exfoliation), yet another improvement over existing numbing creams.

The present disclosure provides compositions for numbing pain during skin-related procedures, the novel formulation being surprisingly and unexpectedly safer and more efficacious than existing topical creams. In embodiments, the composition is a topical cream.

In embodiments, the cream comprises one or more of lidocaine in a range of about 1-30 wt %, prilocaine in a range of about 1-5 wt %, tetracaine in a range of about 1-10 wt %, or benzyl alcohol in a range of about 1-5 wt %. In embodiments, the cream comprises about 1-50 wt % of one or more local anesthetic; about 1-30 wt % of a film-forming agent and/or a co-delivery agent; and about 20-70 wt % water.

In embodiments, the compositions of the present disclosure are removed by peeling following topical administration of the composition to a patient in need thereof. In embodiments, upon topical administration to a patient in need thereof, the compositions of the present disclosure provide a self-occluding layer, which aids in increasing the numbing effect of the cream by minimizing the evaporation of local anesthetics and enhancing the absorption of local anesthetic into the skin. In embodiments, the compositions of the present disclosure dry or cure quickly and peel off to minimize patient discomfort and facilitate removal. In embodiments, the compositions of the present disclosure provide reduced side effect profiles (such as skin irritation) compared to existing numbing creams.

In embodiments, the compositions of the present disclosure comprise a codelivery agent to improve the topical absorption of the local anesthetic. In embodiments, the cream comprises one or more protective agents to reduce skin dryness and irritation. In embodiments, the cream comprises one or more preservatives to provide shelf stability of the product. In embodiments, the cream comprises a solvent as a base. In embodiments, the cream comprises a synthetic polymer as a film-forming and binding agent.

In embodiments, the topical cream comprises about 1-5 wt % of one or more local anesthetic, a codelivery agent, a film-forming agent, and an occlusive agent.

In embodiments, the topical cream comprises about 1-5 wt % of lidocaine and/or prilocaine, a codelivery agent, a film-forming agent, and an occlusive agent.

In embodiments, the topical cream comprises about 1-30 wt % lidocaine, about 1-10 wt % of tetracaine, a codelivery agent, a film-forming agent, and an occlusive agent.

In embodiments, the topical cream comprises one or more wound healing and anti-microbial agents. The use of wound healing and anti-microbial agents can reduce infection, protect the wound site, and improve healing outcomes. In embodiments, the cream dries or cures into a liquid bandage with numbing, wound healing, and/or anti-microbial properties.

In embodiments, the cream comprises about 1-5 wt % of lidocaine and/or prilocaine, codelivery agent, a film-forming agent, and an occlusive agent.

In embodiments, a cream is provided that comprises wound healing and anti-microbial agents that can reduce infection, protect the wound site, and improve healing outcomes. In embodiments, the cream will dry into a liquid bandage with numbing, wound healing, and/or anti-microbial properties. In embodiments, the cream comprises about 1-30 wt % of lidocaine, about 1-10 wt % of tetracaine, a codelivery agent, a film-forming agent, and an occlusive agent.

Aspects of the invention include Aspect 1, which is a composition comprising: 1-50 wt % of one or more local anesthetic; 1-30 wt % of one or more film-forming agent and/or one or more co-delivery agent; and 20-70 wt % water and/or one or more additional components.

Aspect 2 is a composition comprising: i) about 1-10 wt % or about 10-35 wt % of one or more local anesthetic; ii) about 1-25 wt % of one or more co-delivery agent or about 1-30 wt % of one or more film-forming agent; and iii) 20-70 wt % water and/or one or more additional components. In embodiments, the composition further comprises one or more of the additional components in about 1-10 wt % of one or more occlusive agent and/or one or more color-changing/indicating agent.

Aspect 3 is the composition of Aspects 1 or 2, wherein the local anesthetic is chosen from lidocaine or a pharmaceutically acceptable salt thereof, prilocaine or a pharmaceutically acceptable salt thereof, tetracaine or a pharmaceutically acceptable salt thereof, benzyl alcohol, or a mixture thereof.

Aspect 4 is the composition of any of Aspects 1-3, wherein: the local anesthetic is lidocaine or a pharmaceutically acceptable salt thereof. In embodiments, the local anesthetic is tetracaine or a pharmaceutically acceptable salt thereof. In embodiments, the local anesthetic is prilocaine or a pharmaceutically acceptable salt thereof. In embodiments, the local anesthetic is benzyl alcohol. In embodiments, the local anesthetic is lidocaine or a pharmaceutically acceptable salt thereof and tetracaine or a pharmaceutically acceptable salt thereof. In embodiments, the local anesthetic is lidocaine or a pharmaceutically acceptable salt thereof and prilocaine or a pharmaceutically acceptable salt thereof. In embodiments, the local anesthetic is lidocaine or a pharmaceutically acceptable salt thereof and benzyl alcohol. In embodiments, the local anesthetic is tetracaine or a pharmaceutically acceptable salt thereof and prilocaine or a pharmaceutically acceptable salt thereof. In embodiments, the local anesthetic is tetracaine or a pharmaceutically acceptable salt thereof and benzyl alcohol. In embodiments, the local anesthetic is prilocaine or a pharmaceutically acceptable salt thereof and benzyl alcohol. In embodiments, the local anesthetic is lidocaine or a pharmaceutically acceptable salt thereof, tetracaine or a pharmaceutically acceptable salt thereof and prilocaine or a pharmaceutically acceptable salt thereof. In embodiments, the local anesthetic is lidocaine or a pharmaceutically acceptable salt thereof, tetracaine or a pharmaceutically acceptable salt thereof, prilocaine or a pharmaceutically acceptable salt thereof and benzyl alcohol, or a mixture thereof.

Aspect 5 of the present disclosure is a composition comprising about 1-10 wt % of an occlusive agent.

Aspect 6 of the present disclosure is a composition comprising antimicrobials and/or antiseptics. In embodiments, the composition comprises ethanol.

Aspect 7 of the present disclosure is a composition comprising one or more of the co-delivery agents. In embodiments, one or more of the co-delivery agents is glycerin.

Aspect 8 of the present disclosure is a composition comprising one or more of the film-forming agents. In embodiments, the one or more film-forming agents is polyvinylpyrrolidone, polyvinyl alcohol, potassium sorbate, carbopol, xanthan gum, or hydroxypropyl methylcellulose.

Aspect 9 of the present disclosure is a composition comprising one or more of the occlusive agents, such as, by way of example, cosmetic wax, such as paraffin wax or beeswax.

Aspect 10 of the present disclosure is a composition comprising one or more of the additional components, wherein the one or more additional components can be a color-changing agent.

Aspect 11 of the present disclosure is a composition wherein one or more of the additional components are chosen from emulsifiers, emollients, and/or preservatives, or a mixture thereof.

Aspect 12 of the present disclosure is a composition wherein one or more of the additional components are wound-healing agents, menthol, protective agents and/or scar-reducing/preventing agents.

Aspect 13 of the present disclosure is a composition wherein one or more of the additional components are protective agents, such as about 1-10 wt % of pantothenic acid or panthenol.

Aspect 14 of the present disclosure is a composition wherein one or more of the co-delivery agents is: Aloe vera; Beeswax; Caprylic/capric triglyceride; Cetyl alcohol; Emulsifying wax; Glycerin; Hyaluronic acid; Jojoba oil; Lecithin; Mineral oil; Olive oil; Phospholipids; Shea butter; Silicone oil; Sorbitan stearate; Squalane; Stearic acid; Sunflower seed oil; Tocopherol (vitamin E); Urea; Vitamin C (ascorbic acid); Vitamin A (retinol); Witch hazel; Xanthan gum; Zinc oxide, or a mixture thereof.

Aspect 15 of the present disclosure is a composition wherein one or more of the film-forming agents is Acacia Senegal Gum; Acrylates Copolymer; Algin; Carbomer; Carrageenan; Cellulose; Chitosan; Ethyl cellulose; Gelatin; Hydroxyethylcellulose; Pectin; Polyvinyl Alcohol; polyvinylpyrrolidone; Pullulan; Sodium Alginate; Sodium Carboxymethylcellulose; Xanthan Gum; Benzyl alcohol; Butylene glycol; Caprylyl glycol; Ethylhexylglycerin; Phenoxyethanol; Potassium sorbate; Sodium benzoate; Sodium dehydroacetate; Sodium metabisulfite; and Tetrasodium EDTA, or a mixture thereof.

Aspect 16 of the present disclosure is a composition wherein one or more of the additional components is alcohol; Benzalkonium chloride; Benzethonium chloride; Cetylpyridinium chloride; Chlorhexidine; Ethanol (ethyl alcohol); Hydrogen peroxide; Iodine; Isopropyl alcohol; Methylparaben; Neomycin sulfate; Povidone-iodine; Silver sulfadiazine; Tea tree oil (Melaleuca alternifolia); Triclosan; and Zinc oxide; Allantoin; Aloe vera; Antioxidants (e.g., vitamin E, green tea extract); Beeswax; Biotin (Provitamin or Vitamin B7); Calendula extract; Ceramides; Chamomile extract; Cobalamin (Provitamin or Vitamin B12); Folic acid or folate (Provitamin or Vitamin B9); Glycerin; Hyaluronic acid; Jojoba oil; Lanolin; Niacinamide or Nicotinic Acid (Provitamin or Vitamin B3); Oat extract; Panthenol or Pantothenic Acid (Provitamin or Vitamin B5): Peptides; Pyridoxine (Provitamin or Vitamin B6); Riboflavin (Provitamin or Vitamin B2); Shea butter; Sunflower seed oil; Thiamine (Provitamin or Vitamin B1); Titanium dioxide (for sun protection); Zinc oxide (for sun protection), or a mixture thereof.

Aspect 17 of the present disclosure is a composition wherein one or more of the occlusive agents is Beeswax; Candelilla wax; Carnauba wax; Acetyl alcohol; Lanolin; Mineral oil; Ozokerite; Paraffin wax; Petrolatum; Shea butter; Squalane; Stearyl alcohol; Synthetic beeswax; Synthetic wax, or a mixture thereof.

### BRIEF DESCRIPTION OF THE DRAWINGS

The accompanying drawings illustrate certain aspects of some of the embodiments of the present invention and should not be used to limit or define the invention. Together with the written description the drawings serve to explain certain principles of the invention.

FIG. 1 illustrates a self-occlusive property of the cream of the Example 1. The formation of the solid and occlusive layer over the area of application after the drying and curing of the cream as described in Example 1 is shown.

FIG. 2 illustrates a function of a codelivery agent(s) in enhancing the absorption of the local anesthetics into the skin according to embodiments of the present disclosure.

FIG. 3 demonstrates an application and peel off according to embodiments of the present disclosure. Upon application, the cream will dry or cure to form a flexible occlusive layer on top of the skin. Once the film has formed, it will remain on the skin for a period of time to induce the necessary level of numbing. The film can then be peeled off by, for example, grasping the edges and pulling the layer away from the skin. Upon the application and removal of the numbing cream, the top layer of the skin will present with biochemical changes or mechanical disruption.

### DETAILED DESCRIPTION

### Definitions

Throughout this disclosure, various patents, patent applications and publications (including non-patent publications) are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided in the present disclosure, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties for all purposes and as such are intended to provide an efficient way of supplementing the enabling disclosure of this invention as well as provide background detailing the level of ordinary skill in the art.

The term “about” when immediately preceding a numerical value means a range of plus or minus 5% of that value. For example, “about 50” can mean 47.5 to 52.5, etc., unless the context of the disclosure indicates otherwise, or is inconsistent with such an interpretation.

As used herein, the term “patient” generally describes the human or animal to whom the cream is being applied to, whether in a clinical or over-the-counter setting.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. The nature of the salt is not critical, provided that it is pharmaceutically acceptable.

As used herein, wt % as used herein refers to wt. % per unit volume of the composition.

It is to be understood that while certain of the illustrations and figure may be close to the right scale, most of the illustrations and figures are not intended to be of the correct scale.

Furthermore, it is to be understood that the invention can be carried out or practiced in various ways and that the invention can be implemented in embodiments other than the ones outlined in the description above.

The present disclosure, among other things, provides compositions (such as a topical cream) for numbing pain during dermatology procedures.

In embodiments, the compositions of the present disclosure provide reduced toxicity. In embodiments, the compositions of the present disclosure are packaged into an improved packaging which limits the amount of product dispensed from the dispending device based on a pre-determined amount needed for particular part of body, such as on the face, the neck, or the body. In embodiments, the dispensing device limits or controls how much of the topical cream, and therefore local anesthetic(s) in the topical cream, is applied. In embodiments, the effective amount limits and controls numbing effect at the treatment site.

In embodiments, the topical cream formulating further comprises ingredients that cause the product to change color upon application and drying (i.e., color-changing agent). In embodiments, the color-changing agent are any one or more of the following:

### Color-Indicating Pigments or Dyes:

Red 27 (CI 45410)
Red 33 (CI 17200)
Blue 1 (CI 42090)
Blue 27 (CI 73015)
Yellow 5 (CI 19140)
Yellow 6 (CI 15985)

And other variations or chemicals.

### Photochromic Compounds:

Spiropyran derivatives
Diarylethene derivatives
   pH-Responsive Colorants:
Anthocyanins (e.g., from fruits like blueberries, grapes, or cherries)
Red cabbage extract (changes color depending on pH)

### Thermochromic Compounds:

Leuco dyes

### Time-Dependent Colorants:

Decomposable colorants or dyes that gradually change color over time (such as certain natural colorants or synthetic compounds)

### Encapsulated Color-Changing Particles:

Microcapsules containing colorants that release pigments upon certain triggers (e.g., absorption, temperature, time, or shining of a light)
Non-fatty acid lipid coloring agents that react with the top layer of the skin to change colors upon application.

In embodiments, the composition of the present disclosure changes color when topically administered on the skin of a subject in need thereof. In embodiments, the color of the cream after application changes based on the rate of active ingredient absorption into the skin or tissue. In embodiments, the color of the cream after application changes based on, for example based on how long the topical cream has been applied to the skin or tissue. Accordingly, in embodiments, the color change provides a metric for understanding a subjective experience of a patient to whom the cream has been applied. In embodiments, the color change allows for greater control over how the product is used across different people applying the product, thereby enhancing its safety and efficacy by improving accurate and proper application of the product. In other words, the color changing property allows for consistent application of the product regardless of who is applying the product. This allows for more optimal or more preferable application based on, in examples, pre-determined times and/or amounts that should be applied to particular places on a face or body. For example, it may be determined that an identical composition should be applied on the upper lip for a set amount of time, while it has also been determined that that same cream composition should be applied on the forehead for a different amount of time. The times can be, in aspects, keyed to different colors, so the person applying the cream will know to remove or peel-off the cream once it changes to a particular color.

In embodiments, the present disclosure provides a topical cream composition comprising at least one local anesthetic. In embodiments, the composition comprises about 10 wt % to about 30 wt % of lidocaine or a pharmaceutically acceptable salt thereof, for example, about 10-15 wt %, about 15-20 wt %, about 20-25 wt %, about 25-30 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, or about 30 wt %, including all values and ranges therebetween.

In embodiments, the composition can include jojoba or wax (e.g., paraffin wax) as a carrier for enhanced functionality and aiding in delivery of one or more active ingredients. In embodiments, a wax, such as paraffin wax or an alternative cosmetic wax, can be used to improve self-occlusive properties, which can be used to produce a film at or near the top of the cream after application, thereby acting as a film-forming agent to create a layer that reduces evaporation of one or more local numbing agents in the composition (e.g., acting as an occlusive agent). For example, any one or more cosmetic wax can be combined with any one or more film-forming agent to provide for enhanced film-forming and occlusive effects. In aspects, an occlusive agent in the composition can act as a one-way occlusive agent, reducing the ability of ingredients to evaporate. In aspects, an occlusive agent in the composition can act as a one-way occlusive agent, reducing the ability of outside influences penetrating into the cream after application. In aspects, an occlusive agent in the formulation can act as a two-way occlusive agent, reducing the ability of outside influences penetrating into the cream after application and reducing evaporation of one or more local numbing agents in the composition.

In embodiments, the present disclosure provides a topical cream composition comprising lidocaine or a pharmaceutically acceptable salt thereof at a concentration of about 1-5 wt % (for example, about 1 wt %, about 2 wt %, about 3 wt %, about 4 wt %, about 5 wt %, about 1-2 wt %, about 2-3 wt %, about 3-4 wt %, or about 4-5 wt % including all values and ranges therebetween).

In embodiments, the present disclosure provides a topical cream comprises lidocaine or a pharmaceutically acceptable salt thereof and prilocaine or a pharmaceutically acceptable salt thereof, each at concentrations of about 1-5 wt % (for example, about 1 wt %, about 2 wt %, about 3 wt %, about 4 wt %, about 5 wt %, about 1-2 wt %, about 2-3 wt %, about 3-4 wt %, or about 4-5 wt % including all values and ranges therebetween) for over-the-counter use. In embodiments, the composition comprises lidocaine or a pharmaceutically acceptable salt thereof and prilocaine or a pharmaceutically acceptable salt thereof, a film-forming agent, a codelivery agent, a protective agent, and preservatives.

In embodiments, the present disclosure provides a topical cream comprising lidocaine or a pharmaceutically acceptable salt thereof, at a concentration of about 1-30 wt % (for example, about 1-5 wt %, about 5-10 wt %, about 10-15 wt %, about 15-20 wt %, about 20-25 wt %, or about 25-30 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, including all values and ranges therebetween) and tetracaine or a pharmaceutically acceptable salt thereof at a concentration of about 1-10 wt % (for example, about 1-2 wt %, about 2-3 wt %, about 3-4 wt %, about 4-5 wt %, about 1 wt %, about 2 wt %, about 3 wt %, about 4 wt %, about 5 wt %, about 6 wt %, about 7 wt %, about 8 wt %, about 9 wt %, about 10 wt %, including all values and ranges therebetween). In embodiments, the composition comprises lidocaine or a pharmaceutically acceptable salt thereof and tetracaine or a pharmaceutically acceptable salt thereof, a film-forming agent, a codelivery agent, a protective agent, and preservatives. In embodiments, the composition is manufactured for use in a clinical setting.

In embodiments, a topical cream is provided comprising lidocaine in a concentration between 1-5 wt %, such as from 1-2 wt %, 2-3 wt %, 3-4 wt %, and 4-5 wt %. This formulation can include lidocaine, a film-forming agent, a codelivery agent, a protective agent, preservatives, and other ingredients. This composition can include wound healing and anti-microbial agents to help reduce infection, protect the wound site, and to improve healing outcomes. In this embodiment, the cream can dry into a liquid bandage with numbing, wound healing, and/or anti-microbial properties.

In embodiments, the topical cream compositions of the present disclosure comprises lidocaine or a pharmaceutically acceptable salt thereof and prilocaine or a pharmaceutically acceptable salt thereof each in concentrations of about 1-5 wt % (for example, about 1 wt %, about 2 wt %, about 3 wt %, about 4 wt %, about 5 wt %, about 1-2 wt %, about 2-3 wt %, about 3-4 wt %, or about 4-5 wt % including all values and ranges therebetween). In embodiments, the topical cream composition comprises lidocaine or a pharmaceutically acceptable salt thereof and prilocaine or a pharmaceutically acceptable salt thereof, a film-forming agent, a codelivery agent, a protective agent, and preservatives. In embodiments, the topical cream composition further comprises wound healing and anti-microbial agents.

In embodiments, the present disclosure provides the use of an external system or mechanism to dry or cure the cream.

In embodiments, the compositions and creams according to the current disclosure can dry into a liquid bandage with numbing, wound healing, and/or anti-microbial properties.

In embodiments, the present disclosure provides the use of a naturally-derived film-forming agent(s).

In embodiments, the present disclosure provides the use of some or only biodegradable ingredients. In embodiments, the composition of the present disclosure is biodegradable.

In embodiments, the present disclosure provides the use of an external system or mechanism to dry or cure the cream.

In embodiments, the present disclosure provides the use of one or more additional topical layer applied over the numbing cream itself.

In embodiments, the present disclosure provides the use of wound-healing agents for use over open wounds such as burn wounds or cuts.

In embodiments, the present disclosure provides the use of the product in hair removal products, such as wax or sugar.

In embodiments, the present disclosure provides the use of the product on animals.

In embodiments, the compositions of the present disclosure further comprise a film-forming agent, a codelivery agent, a protective agent, preservatives. In embodiments, the composition is manufactured for over-the-counter use. In embodiments, the present disclosure relates to a topical cream composition providing the highest concentration of local anesthetics in accordance with regulatory guidelines. In embodiments, the compositions of the present disclosure also dry quickly, reducing inconveniences to the patient and allowing the patient to move about freely during the waiting period. The compositions of the present disclosure, in embodiments, self-occlude, which eliminate the need to wrap the cream with any additional materials and enhances the absorption of the numbing particles into the skin for a stronger numbing effect. The compositions of the present disclosure comprise ingredients selected to minimize side effect profiles to reduce reports of skin irritation or allergic reactions. In embodiments, the compositions of the present disclosure peel off, eliminating the need to wipe off the product. In embodiments, the topical cream composition further comprises wound healing and anti-microbial agents to help reduce infection, protect the wound site, and to improve healing outcomes. In embodiments, the cream composition dries into a liquid bandage with numbing, wound healing, and/or anti-microbial properties.

In embodiments, the topical cream compositions are in different colors, for example, for pediatric and athletic purposes. In embodiments, the topical cream composition will include a cooling ingredient, such as menthol.

In embodiments, the topical cream composition further comprises jojoba or wax (e.g., paraffin wax) as a carrier for enhanced functionality and aiding in delivery of one or more active ingredients. In embodiments, a wax, such as paraffin wax or an alternative cosmetic wax, is used to improve self-occlusive properties, which is used to produce a film at or near the top of the cream after application, thereby acting as a film-forming agent to create a layer that reduces evaporation of one or more local anesthetics in the composition (e.g., acting as an occlusive agent). In embodiments, one or more cosmetic waxes are combined with one or more film-forming agents to provide enhanced film-forming and occlusive effects. In embodiments, the occlusive agent in the composition acts as a one-way occlusive agent, reducing the ability of ingredients to evaporate. In embodiments, the occlusive agent in the composition acts as a one-way occlusive agent, reducing the ability of outside influences penetrating into the cream composition after application. In embodiments, the occlusive agent in the composition acts as a two-way occlusive agent, reducing the ability of outside influences penetrating into the cream composition after application and reducing evaporation of one or more local anesthetics in the composition.

In embodiments, the cream is formulated only with naturally degrading products so that the cream can biodegrade. In embodiments, the cream is formulated using only plant-derived ingredients.

In embodiments, the occlusive agent for cream compositions is Beeswax; Candelilla wax; Carnauba wax; Acetyl alcohol; Cetearyl alcohol; Palm oil; fatty alcohols; Lanolin; Mineral oil; Ozokerite; Paraffin wax; Petrolatum; Shea butter; Squalane; Stearyl alcohol; Synthetic beeswax; or Synthetic wax, or a mixture thereof.

In embodiments, the film-forming agent is Acacia Senegal Gum; Acrylates Copolymer; Algin; Carbomer; Carrageenan; Cellulose; Chitosan; Ethyl cellulose; Gelatin; Hydroxyethylcellulose; Pectin; Polyvinyl Alcohol; polyvinylpyrrolidone; Pullulan; Sodium Alginate; Sodium Carboxymethylcellulose; or Xanthan Gum, or a mixture thereof.

In embodiments, the film-forming agent is Benzyl alcohol; Butylene glycol; Caprylyl glycol; Ethylhexylglycerin; Phenoxyethanol; Potassium sorbate; Sodium benzoate; Sodium dehydroacetate; Sodium metabisulfite; or Tetrasodium EDTA, or a mixture thereof.

In embodiments, the co-delivery agent is Aloe vera gel; Beeswax; Caprylic/capric triglyceride; Cetyl alcohol; Emulsifying wax; Glycerin; Hyaluronic acid; Jojoba oil; Lecithin; Mineral oil; Olive oil; Phospholipids; Shea butter; Silicone oil; Sorbitan stearate; Squalane; Stearic acid; Sunflower seed oil; Tocopherol (vitamin E); Urea; Vitamin C (ascorbic acid); Vitamin A (retinol); Witch hazel extract; Xanthan gum; or Zinc oxide, or a mixture thereof. In embodiments, the composition comprises 0.5 wt % to about 20 wt % of one or more co-delivery agent(s), such about 0.5, 1, 2, 3, 4, 5, 6, 7, 8, 9, 10, 11, 12, 13, 14, 15, 16, 17, 18, or 19 wt %, including all values and ranges therebetween.

In embodiments, the protective agent for cream compositions described herein is Allantoin; Aloe vera; Antioxidants (e.g., vitamin E, green tea extract); Beeswax; Biotin (Provitamin or Vitamin B7); Calendula extract; Ceramides; Chamomile extract; Cobalamin (Provitamin or Vitamin B12); Folic acid or folate (Provitamin or Vitamin B9); Glycerin; Hyaluronic acid; Jojoba oil; Lanolin; Niacinamide or Nicotinic Acid (Provitamin or Vitamin B3); Oat extract; Panthenol or Pantothenic Acid (Provitamin or Vitamin B5): Peptides; Pyridoxine (Provitamin or Vitamin B6); Riboflavin (Provitamin or Vitamin B2); Shea butter; Sunflower seed oil; Thiamine (Provitamin or Vitamin B1); Titanium dioxide (for sun protection); Zinc oxide (for sun protection), or a mixture thereof. In embodiments, one or more protective agent(s) is/are included in the composition, wherein each protective agent is included at a concentration of up to about 20 wt %, such as up to about 0.5, 1, 2, 3, 4, 5, 6, 7, 8, 9, 10, 11, 12, 13, 14, 15, 16, 17, 18, or 19 wt %, including all values and ranges therebetween.

In embodiments, to create a peeling-off aspect of the creams of the disclosure as described herein, ingredients are used to promote exfoliation and/or help the cream peel off the skin. In embodiments, the composition comprises about 0.5 wt % to about 20 wt % of one or more peeling agent(s), such as about 0.5, 1, 2, 3, 4, 5, 6, 7, 8, 9, 10, 11, 12, 13, 14, 15, 16, 17, 18, or 19 wt %, including all values and ranges therebetween. In embodiments, the peeling agent is one or more of the following:
a. Alpha hydroxy acids (AHAs): Examples include glycolic acid, lactic acid, citric acid, malic acid, and tartaric acid. AHAs are known for their exfoliating properties and can help remove dead skin cells.
b. Bentonite clay: This natural clay has absorbent properties and can help draw out impurities from the skin, contributing to the peeling effect.
c. Enzymes: Proteolytic enzymes such as papain (derived from papaya) or bromelain (derived from pineapple) can break down proteins and facilitate the removal of dead skin cells.
d. Kaolin clay: Similar to bentonite clay, kaolin clay has absorbent qualities and can help in the removal of impurities and dead skin cells.
e. Polyvinylpyrrolidone (PVP): A synthetic polymer that creates uniform, thin films on skin and hair. It can serve as a film-forming agent and/or a thickener.
f. Polyvinyl alcohol (PVA): This synthetic polymer forms a film when it dries, allowing it to be peeled off along with impurities and dead skin cells.
g. Salicylic acid: This beta hydroxy acid (BHA) has exfoliating properties and can penetrate the pores to help remove excess oil and dead skin cells, promoting a peeling effect.
h. Silicone: Silicone-based ingredients can create a film on the skin, facilitating the peeling process when dry.
i. Tretinoin: A derivative of vitamin A, tretinoin promotes cell turnover and can aid in the removal of dead skin cells.
j. Agents that assist with the peeling off process can also include one or more of the following, including agents that help with reducing irritation at the area where the hardened cream is pulled off, including but not limited to: glycerin, jojoba, and paraffin wax.
k. Wound healing agents is one or more of the following: Aloe vera: Known for its soothing and moisturizing properties, aloe vera promotes wound healing and reduces inflammation.
l. Calendula: Possessing anti-inflammatory and antimicrobial properties, calendula helps heal wounds, burns, and cuts.
m. Chamomile: Chamomile has anti-inflammatory and antimicrobial effects, making it useful for wound healing and reducing skin irritation.
n. Honey: With its antibacterial properties, honey promotes wound healing and reduces the risk of infection. (ex. Manuka honey)
o. Jojoba oil: Jojoba oil moisturizes the skin and aids in wound healing due to its antimicrobial properties.
p. Lavender oil: Lavender oil possesses antibacterial and anti-inflammatory properties, making it beneficial for wound healing.
q. Neem oil: Neem oil has antibacterial and antifungal properties, helping to prevent infections and promote wound healing.
r. Rosehip oil: Rich in essential fatty acids and antioxidants, rosehip oil aids in tissue regeneration and reduces the appearance of scars.
s. Tea tree oil: Tea tree oil has antimicrobial properties, making it effective in preventing infections and promoting wound healing.
t. Vitamin E: Vitamin E is an antioxidant that helps protect the skin from damage and supports wound healing.

In embodiments, the composition comprises about 0.5 wt % to about 20 wt % of one or more anti-microbial agent(s), such as about 0.5, 1, 2, 3, 4, 5, 6, 7, 8, 9, 10, 11, 12, 13, 14, 15, 16, 17, 18, or 19 wt %, including all values and ranges there between. In embodiments, the anti-microbial agents is Benzalkonium chloride; Benzethonium chloride; Cetylpyridinium chloride; Chlorhexidine; Ethanol (ethyl alcohol); Hydrogen peroxide; Iodine; Isopropyl alcohol; Methylparaben; Neomycin sulfate; Povidone-iodine; Silver sulfadiazine; Tea tree oil (Melaleuca alternifolia); Triclosan; Zinc oxide, or a mixture thereof.

The liquid bandage ingredients are selected from one or more of the following:
a. Acrylates Copolymer: A film-forming agent that creates a protective barrier over the skin.
b. Alcohol: Used as a solvent and to promote quick drying.
c. Benzoin Tincture: An antiseptic and adhesive agent.
d. Butylated Hydroxytoluene (BHT): An antioxidant that helps prevent the cream from spoiling.
e. Castor Oil: Provides a smooth texture and aids in adhesion.
f. Ethylhexyl Palmitate: A lightweight emollient that enhances spreadability.
g. Isopropyl Alcohol: Helps in quick drying and acts as a disinfectant.
h. Liquid Paraffin: Forms a protective layer on the skin and helps retain moisture.
i. Petrolatum: Acts as an occlusive agent to seal the wound and protect it from external irritants.
j. Polyethylene Glycol: A thickening agent that improves the cream's texture.
k. Silicone Oil: Enhances spreadability and creates a flexible film over the skin.
l. Zinc Oxide: Provides a drying effect and promotes wound healing.

In embodiments, the compositions of the present disclosure further comprises Coconut oil, chamomile oil, licorice root extract, evodia, sodium pyrrolidone carboxylic acid, argan oil, evening primrose oil, grapeseed oil, monoi oil, bamboo, coconut water, neroli oil, carrot extract, turmeric, willow bark extract, algae extract, macadamia nut oil, rosemary oil, behenic acid, ceramide NS, ceramide EOP, ceramide EOS, cucumber fruit extract, hydrolyzed rice protein, algae-derived extracts, ginger root extract, Betula alba extract, witch hazel, disodium EDTA, or a mixture thereof.

In embodiments, the topical cream compositions of the present disclosure further comprise anesthetics, co-delivery agents, occlusive agents, film-forming agents, peeling agents, and skin protective agents. In embodiments, the topical cream compositions of the present disclosure further comprise additional components including, but not limited to, one or more emulsifiers, emollients, preservatives, color-changing agents, wound-healing agents, scar-preventing agents, antimicrobials/antiseptics, or solvents, or a mixture thereof. In embodiments, the topical cream compositions of the present disclosure further comprise water or alcohol (such as ethanol or isopropyl alcohol or the like) alone.

In embodiments, the topical cream compositions of the present disclosure further comprise about 1-10 wt % menthol, such as about 2, 3, 4, 5, 6, 7, 8 or 9 wt % menthol, including all values and ranges therebetween.

In embodiments, the topical creams of the present disclosure further comprise one or more wound-healing agents.

In embodiments, the topical creams of the present disclosure further comprise a color-changing agents.

In embodiments, the topical creams of the present disclosure further comprise about 1-5 wt % of potassium sorbate, such as about 2, 3, or 4 wt %, including all values and ranges therebetween.

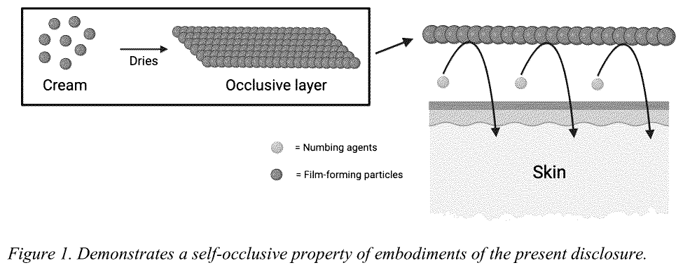

Turning now to the figures, FIG. 1 shows an example of a self-occlusive property of embodiments according to the present invention. In those embodiments, once applied, the cream can dry or cure; in cases, the applied cream will dry or cure over a short period of time, such as under one second, from one second to two seconds, from two seconds to three seconds, from three seconds to four seconds, from four seconds to five seconds, from five seconds to six seconds, and so on. In aspects, a synthetic polymer and an occluding agent will form a solid and occlusive layer over the area of application, minimizing the evaporation of numbing agents and enhancing the absorption of numbing agent(s) into the skin.

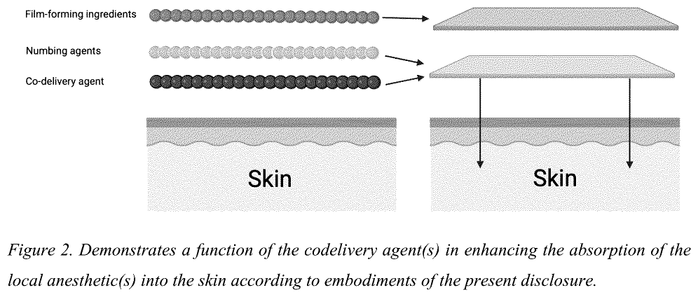

FIG. 2 shows a function of codelivery agents according to embodiments of the present invention, whereby absorption of the numbing agent(s) is aided by a codelivery agent. Codelivery agents, in aspects, can couple with the numbing agent(s) once the cream is applied, improving the mechanism of action and increasing absorption of numbing agent(s) into the skin.

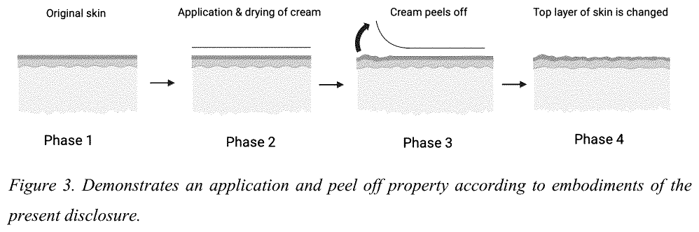

FIG. 3 shows a peel-off property according to embodiments of the present invention. In those embodiments, upon application, the cream will dry or cure to form a flexible occlusive layer on top of the skin. Once the film has formed, it will remain on the skin for a period of time to induce the necessary level of numbing. The film can then be peeled off by, for example, grasping the edges and pulling the layer away from the skin. Upon the application and removal of the numbing cream, the top layer of the skin will present with biochemical changes or mechanical disruption.

### NON-LIMITING EXAMPLES OF FORMULATIONS

### Example 1: Compositions

The components of the exemplary topical cream compositions of the present disclosure are summarized below.

| Embodiments | Formulation A | Formulation B |
|---|---|---|
| Base | 1-5% Lidocaine | 10-23% Lidocaine |
| | 1-5% Prilocaine | 1-10% Tetracaine |
| | 1-10% Glycerin | 1-10% Glycerin |
| | 30-70% Water | 30-70% Water |
| | 5-18% Polyvinylpyrrolidone | 5-18% Polyvinylpyrrolidone |
| | 1-10% Ethanol | 1-10% Ethanol |
| | 1-5% Potassium Sorbate | 1-5% Potassium Sorbate |
| | Emulsifiers | Emulsifiers |
| | Emollients | Emollients |
| | Preservatives | Preservatives |
| | Color-changing agent (time or | Color-changing agent (time or |
| | absorption) | absorption) |
| In this embodiment, aloe vera | 1-5% Lidocaine | 10-23% Lidocaine |
| and pantothenic acid are | 1-5% Prilocaine | 1-10% Tetracaine |
| added to minimize skin | 1-10% Glycerin | 1-10% Glycerin |
| irritation, improve hydration | 30-70% Water | 30-70% Water |
| and care of skin, and to reduce | 5-18% Polyvinylpyrrolidone | 5-18% Polyvinylpyrrolidone |
| inflammation and redness. | 1-10% Ethanol | 1-10% Ethanol |
| | 1-5% Potassium Sorbate | 1-5% Potassium Sorbate |
| | 1-15% Aloe vera | 1-15% Aloe vera |
| | 1-10% Pantothenic acid | 1-10% Pantothenic acid |
| | Emulsifiers | Emulsifiers |
| | Emollients | Emollients |
| | Preservatives | Preservatives |
| | Color-changing agent (time or | Color-changing agent (time or |
| | absorption) | absorption) |
| In this embodiment, menthol is | 1-5% Lidocaine | 10-23% Lidocaine |
| added to provide a mild | 1-5% Prilocaine | 1-10% Tetracaine |
| cooling effect. | 1-10% Glycerin | 1-10% Glycerin |
| | 30-70% Water | 30-70% Water |
| | 5-18% Polyvinylpyrrolidone | 5-18% Polyvinylpyrrolidone |
| | 1-10% Ethanol | 1-10% Ethanol |
| | 1-5% Potassium Sorbate | 1-5% Potassium Sorbate |
| | 1-15% Aloe vera | 1-15% Aloe vera |
| | 1-10% Pantothenic acid | 1-10% Pantothenic acid |
| | Emulsifiers | Emulsifiers |
| | Emollients | Emollients |
| | Preservatives | Preservatives |
| | 1-10% Menthol | 1-10% Menthol |
| | Color-changing agent (time or | Color-changing agent (time or |
| | absorption) | absorption) |
| In this embodiment, wound | 1-5% Lidocaine | 10-23% Lidocaine |
| healing agents are added to | 1-5% Prilocaine | 1-10% Tetracaine |
| improve the recovery of the | 1-10% Glycerin | 1-10% Glycerin |
| skin following injury or minor | 30-70% Water | 30-70% Water |
| medical procedures. | 5-18% Polyvinylpyrrolidone | 5-18% Polyvinylpyrrolidone |
| | 1-10% Ethanol | 1-10% Ethanol |
| | 1-5% Potassium Sorbate | 1-5% Potassium Sorbate |
| | 1-15% Aloe vera | 1-15% Aloe vera |
| | 1-10% Pantothenic acid | 1-10% Pantothenic acid |
| | Emulsifiers | Emulsifiers |
| | Emollients | Emollients |
| | Preservatives | Preservatives |
| | 1-10% Menthol | 1-10% Menthol |
| | Wound Healing Agents | Wound Healing Agents |
| | Color-changing agent (time or | Color-changing agent (time or |
| | absorption) | absorption) |
| In this embodiment, cosmetic | 1-5% Lidocaine | 10-23% Lidocaine |
| wax is added to improve the | 1-5% Prilocaine | 1-10% Tetracaine |
| film-forming and self- | 1-10% Glycerin | 1-10% Glycerin |
| occlusive properties. | 30-70% Water | 30-70% Water |
| | 5-18% Polyvinylpyrrolidone | 5-18% Polyvinylpyrrolidone |
| | 1-10% Ethanol | 1-10% Ethanol |
| | 1-5% Potassium Sorbate | 1-5% Potassium Sorbate |
| | 1-15% Aloe vera | 1-15% Aloe vera |
| | 1-10% Pantothenic acid | 1-10% Pantothenic acid |
| | Emulsifiers | Emulsifiers |
| | Emollients | Emollients |
| | Preservatives | Preservatives |
| | 1-10% Menthol | 1-10% Menthol |
| | Wound Healing Agents | Wound Healing Agents |
| | Color-changing agent (time or | Color-changing agent (time or |
| | absorption) | absorption) |
| | 1-10% Cosmetic wax | 1-10% Cosmetic wax |
| In this embodiment, scar- | 1-5% Lidocaine | 10-23% Lidocaine |
| preventing agents are added | 1-5% Prilocaine | 1-10% Tetracaine |
| to reduce scarring following | 1-10% Glycerin | 1-10% Glycerin |
| injury or a minor medical | 30-70% Water | 30-70% Water |
| procedure. Additionally, | 5-18% Polyvinylpyrrolidone | 5-18% Polyvinylpyrrolidone |
| Antimicrobial and antiseptic | 1-10% Ethanol | 1-10% Ethanol |
| agents are added to protect | 1-5% Potassium Sorbate | 1-5% Potassium Sorbate |
| the wound or treatment area. | 1-15% Aloe vera | 1-15% Aloe vera |
| | 1-10% Pantothenic acid | 1-10% Pantothenic acid |
| | Emulsifiers | Emulsifiers |
| | Emollients | Emollients |
| | Preservatives | Preservatives |
| | 1-10% Menthol | 1-10% Menthol |
| | Wound Healing Agents | Wound Healing Agents |
| | Color-changing agent (time or | Color-changing agent (time or |
| | absorption) | absorption) |
| | 1-10% Cosmetic wax | 1-10% Cosmetic wax |
| | Scar-preventing agents | Scar-preventing agents |
| | Antimicrobial/antiseptic | Antimicrobial/antiseptic |
| Other Iterations/Packaging | The packaging will include a | The packaging will include a |
| | mechanism to supply a pre- | mechanism to supply a pre- |
| | determined dose per | determined dose per |
| | pump/squeeze. This will help | pump/squeeze. This will help |
| | determine dosing per | determine dosing per |
| | application easily and reduce | application easily and reduce |
| | toxicity or side effects | toxicity or side effects |
| | associated with using higher | associated with using higher |
| | than the recommended | than the recommended |
| | amount. The cream will also | amount. The cream will also |
| | be designed to possibly be | be designed to possibly be |
| | mostly biodegradable. | mostly biodegradable. |

### Example 2: Base Compositions

Exemplary topical cream compositions of the present disclosure are listed below.

| | | | | Film- | | Remainder: |
|---|---|---|---|---|---|---|
| | | | | forming | | Additional |
| | | | | and/or | Skin- | Components |
| | | Co-delivery | Occlusive | Peeling | protective | (includes |
| Ex | Anesthetic(s) | agent(s) | agent(s) | agent(s) | agent(s) | solvents) |
| No. | (wt %) | (wt %) | (wt %) | (wt %) | (wt %) | (wt %) |
| 1 | Lidocaine (2) | Glycerin (6) | — | PVA (5) | — | Water (30-70) |
| | Prilocaine (3) | | | | | |
| 2 | Lidocaine (1) | Glycerin | — | PVA (12) | — | Water (30-70) |
| | Prilocaine (1) | (10) | | Potassium | | Ethanol (2) |
| | | | | Sorbate (2) | | |
| 3 | Lidocaine (5) | Glycerin (1) | — | PVA (10) | — | Water (30-70) |
| | Prilocaine (4) | | | | | Ethanol (5) |
| 4 | Prilocaine (5) | Glycerin (2) | — | PVA (18) | — | Water (30-70) |
| | | | | | | Ethanol (8) |
| 5 | Lidocaine (3) | Glycerin (8) | — | PVA (7) | — | Water (30-70) |
| | Prilocaine (2) | | | | | Ethanol (1) |
| 6 | Lidocaine (4) | Glycerin (4) | — | PVA (15) | — | Water (30-70) |
| | Prilocaine (2) | | | | | Ethanol (3) |
| 7 | Lidocaine (12) | Glycerin (6) | — | PVA (5) | — | Water (30-70) |
| | Tetracaine (3) | | | | | Ethanol (10) |
| 8 | Lidocaine (10) | Glycerin | — | PVA (12) | — | Water (30-70) |
| | Tetracaine (1) | (10) | | | | Ethanol (2) |
| 9 | Lidocaine (15) | Glycerin (1) | — | PVA (10) | — | Water (30-70) |
| | Tetracaine (4) | | | | | Ethanol (5) |
| 10 | Benzyl | Glycerin (2) | — | PVA (18) | — | Water (30-70) |
| | Alcohol (5) | | | | | Ethanol (8) |
| 11 | Lidocaine (22) | Glycerin (8) | Beeswax (10) | PVA (7) | — | Water (30-70) |
| | Tetracaine (7) | | | | | Ethanol (1) |
| 12 | Lidocaine (20) | Glycerin (4) | Palm Oil (3) | PVA (15) | — | Water (30-70) |
| | Tetracaine (10) | | | Potassium | | Ethanol (3) |
| | | | | Sorbate (5) | | |
| 13 | Lidocaine (1) | Beeswax (8) | — | PVA (18) | — | Water (30-70) |
| | Prilocaine (5) | | | | | Ethanol (8) |
| 14 | Lidocaine (3) | Hyaluronic | — | PVA (7) | — | Water (30-70) |
| | Prilocaine (2) | acid (2) | | | | Ethanol (1) |
| 15 | Lidocaine (4) | Jojoba oil | — | PVA (15) | — | Water (30-70) |
| | Prilocaine (2) | (4) | | | | Ethanol (3) |
| 16 | Lidocaine (12) | Lecithin (6) | — | PVA (5) | — | Water (30-70) |
| | Tetracaine (3) | | | Potassium | | Ethanol (10) |
| | | | | Sorbate (5) | | |
| 17 | Lidocaine (10) | Cetyl | — | PVA (12) | — | Water (30-70) |
| | Tetracaine (1) | alcohol (10) | | | | Ethanol (2) |
| 18 | Lidocaine (15) | Xanthan | — | PVA (10) | — | Water (30-70) |
| | Tetracaine (4) | gum (7) | | | | Ethanol (5) |
| 19 | Lidocaine + | Glycerin (6) | — | PVA (5) + | Aloe Vera | Water (30-70) |
| | Prilocaine + | | | Potassium | (10) | Ethanol (10) |
| | Benzyl | | | Sorbate (5) | Pantothenic | |
| | Alcohol (10) | | | | acid (10) | |
| 20 | Lidocaine (1) | Glycerin | — | — | Aloe Vera | Water (30-70) |
| | Prilocaine (1) | (10) | | | (10) | Ethanol (2) |
| | | | | | Pantothenic | |
| | | | | | acid (5) | |
| 21 | Lidocaine (5) | Glycerin (1) | — | — | Aloe Vera (1) | Water (30-70) |
| | Prilocaine (4) | | | | Pantothenic | Ethanol (5) |
| | | | | | acid (10) | |
| 22 | Lidocaine (1) | Glycerin (2) | Paraffin Wax | — | Aloe Vera | Water (30-70) |
| | Prilocaine (5) | | (5) | | (10) | Ethanol (8) |
| | | | | | Pantothenic | |
| | | | | | acid (5) | |
| 23 | Lidocaine (3) | Glycerin (8) | — | PVA (7) | Aloe Vera | Water (30-70) |
| | Prilocaine (2) | | | | (15) | Ethanol (1) |
| | | | | | Pantothenic | |
| | | | | | acid (5) | |
| 24 | Lidocaine (4) | Glycerin (4) | — | Acrylates | Aloe Vera (2) | Water (30-70) |
| | Prilocaine (2) | | | Copolymer | | Ethanol (3) |
| | | | | (15) | | |
| 25 | Lidocaine (12) | Glycerin (6) | — | PVA (5) | Aloe Vera | Water (30-70) |
| | Tetracaine (3) | | | | (12) | Ethanol (10) |
| 26 | Lidocaine (10) | Glycerin | — | PVA (12) | Aloe Vera (5) | Water (30-70) |
| | Tetracaine (1) | (10) | | | | Ethanol (2) |
| 27 | Lidocaine (15) | Glycerin (1) | — | PVA (10) | Aloe Vera (7) | Water (30-70) |
| | Tetracaine (4) | | | | | Ethanol (5) |
| 28 | Lidocaine (18) | Glycerin (2) | — | — | Aloe Vera | Water (30-70) |
| | Tetracaine (5) | | | | (14) | Ethanol (8) |
| 29 | Lidocaine (22) | Glycerin (8) | — | — | Aloe Vera | Water (30-70) |
| | Tetracaine (7) | | | | (10) | Ethanol (1) |
| | | | | | Zinc Oxide | |
| | | | | | (20) | |
| 30 | Lidocaine (20) | Glycerin (4) | Cosmetic | — | Aloe Vera (6) | Water (30-70) |
| | Tetracaine (10) | | Wax (4) | | | Ethanol (3) |
| 31 | Benzyl | Beeswax (8) | — | Carrageenan | Aloe Vera (5) | Water (30-70) |
| | Alcohol (10) | | | (18) | | Ethanol (8) |
| 32 | Lidocaine (3) | Hyaluronic | — | Cellulose | Vitamin B, C | Water (30-70) |
| | Prilocaine (2) | acid (2) | | (7) | and E (6) | Ethanol (1) |
| 33 | Lidocaine (4) | Jojoba oil | — | PVA (15) | Lanolin (8) | Water (30-70) |
| | Prilocaine (2) | (4) | | | | Ethanol (3) |
| 34 | Lidocaine (12) | Lecithin (6) | — | PVA (5) + | Aloe Vera (3) | Water (30-70) |
| | Tetracaine (3) | | | Acrylates | | Ethanol (10) |
| | | | | Copolymer | | |
| | | | | (5) | | |
| 35 | Lidocaine (10) | Cetyl | Cetearyl | PVA (12) | Aloe Vera | Water (30-70) |
| | Tetracaine (1) | alcohol (10) | alcohol (5) | | (13) | Ethanol (2) |
| 36 | Lidocaine (15) | Xanthan | — | Pullulan | Aloe Vera | Water (30-70) |
| | Tetracaine (4) | gum (7) | | (10) | (12) | Ethanol (5) |
| 37 | Lidocaine (1) | Glycerin (2) | — | Stearyl | — | Water (30-70) |
| | Prilocaine (5) | | | alcohol | | Ethanol (8) |
| | | | | (18) | | |
| 38 | Lidocaine (3) | Glycerin (8) | — | Sodium | — | Water (30-70) |
| | Prilocaine (2) | | | alginate (7) | | Ethanol (1) |
| 39 | Lidocaine (4) | Glycerin (4) | Cosmetic | Chitosan | — | Water (30-70) |
| | Prilocaine (2) | | Wax (1) | (5) | | Ethanol (3) |
| 40 | Lidocaine (12) | Glycerin (6) | Cosmetic | — | — | Water (30-70) |
| | Tetracaine (3) | | Wax (10) | | | Ethanol (10) |
| 41 | Lidocaine (10) | Glycerin | Cosmetic | — | — | Water (30-70) |
| | Tetracaine (1) | (10) | Wax (4) | | | Ethanol (2) |
| 42 | Lidocaine (15) | Glycerin (1) | Cosmetic | — | — | Water (30-70) |
| | Tetracaine (4) | | Wax (3) | | | Ethanol (5) |
| 43 | Lidocaine (2) | Glycerin | Cosmetic | PVA (5) | Aloe Vera (5) | Water (30-70) |
| | Prilocaine (3) | (10) | Wax (5) | | Pantothenic | Ethanol (10) |
| | | | | | acid (10) | |
| 44 | Lidocaine (1) | Glycerin | Cosmetic | PVA (12) | Aloe Vera (5) | Water (30-70) |
| | Prilocaine (1) | (10) | Wax (10) | | Pantothenic | Ethanol (2) |
| | | | | | acid (1) | |
| 45 | Lidocaine (5) | Glycerin (1) | Cosmetic | PVA (10) | Aloe Vera | Water (30-70) |
| | Prilocaine (4) | | Wax (4) | | (15) | Ethanol (5) |
| | | | | | Pantothenic | |
| | | | | | acid (10) | |
| 46 | Lidocaine (1) | Glycerin (2) | Cosmetic | PVA (18) | Aloe Vera | Water (30-70) |
| | Prilocaine (5) | | Wax (10) | | (15) | Ethanol (8) |
| | | | | | Pantothenic | |
| | | | | | acid (1) | |
| 47 | Lidocaine (3) | Glycerin (8) | Cosmetic | Cellulose | Aloe Vera (8) | Water (30-70) |
| | Prilocaine (2) | | Wax (4) | (20) | Pantothenic | Ethanol (1) |
| | | | | | acid (4) | |
| 48 | Lidocaine (12) | Glycerin (6) | Cosmetic | — | Aloe Vera (3) | Water (30-70) |
| | Tetracaine (3) | | Wax (10) | | Pantothenic | Ethanol (10) |
| | | | | | acid (7) | |
| 49 | Lidocaine (10) | Glycerin | Cosmetic | — | Aloe Vera (5) | Water (30-70) |
| | Tetracaine (1) | (10) | Wax (4) | | Pantothenic | Ethanol (2) |
| | | | | | acid (2) | |
| 50 | Lidocaine (15) | Glycerin (1) | Cosmetic | — | Aloe Vera (1) | Water (30-70) |
| | Tetracaine (4) | | Wax (3) | | Pantothenic | Ethanol (5) |
| | | | | | acid (5) | |
| | | | | | Vitamin C | |
| | | | | | (3) | |
| 51 | Lidocaine (5) | Glycerin (6) | Palm Oil + | PVA (10) + | — | Water (30-70) |
| | | | Shea Butter | Potassium | | Ethanol (10) |
| | | | | Sorbate (2) | | |
| 52 | Prilocaine (2) | Glycerin | — | PVA (12) | — | Water (30-70) |
| | | (10) | | | | Ethanol (2) |
| 53 | Lidocaine (20) | Glycerin (1) | — | PVA (10) | — | Water (30-70) |
| | | | | | | Ethanol (5) |
| 54 | Prilocaine (5) | Glycerin (2) | — | PVA (15) | — | Water (30-70) |
| | | | | Potassium | | Ethanol (8) |
| | | | | Sorbate (5) | | |
| 55 | Lidocaine (3) | Glycerin (8) | Petrolatum | PVA (7) | — | Water (30-70) |
| | Prilocaine (2) | | | | | Ethanol (1) |
| 56 | Lidocaine (8) | Glycerin (4) | — | PVA (15) | — | Water (30-70) |
| | Prilocaine (10) | | | | | Ethanol (3) |
| | Tetracaine (2) | | | | | |
| 57 | Tetracaine (3) | Glycerin (6) | — | PVA (5) + | — | Water (30-70) |
| | | | | Potassium | | Ethanol (10) |
| | | | | Sorbate (5) | | |
| 58 | Tetracaine (15) | Glycerin | Petrolatum | PVA (12) | Vitamin C | Water (30-70) |
| | | (10) | | | (2) | Ethanol (2) |
| | | | | | Pantothenic | |
| | | | | | Acid (2) | |
| | | | | | Hyaluronic | |
| | | | | | Acid (5) | |
| 59 | Lidocaine (15) | — | — | PVA (10) | — | Water (30-70) |
| | Tetracaine (4) | | | | | |
| 60 | Lidocaine (18) | Glycerin (2) | — | PVA (5) + | — | Water (30-70) |
| | Tetracaine (5) | | | Acrylates | | Ethanol (8) |
| | | | | Copolymer | | |
| | | | | (5) | | |
| 61 | Lidocaine (22) | | Beeswax (10) | PVA (7) | — | Water (30-70) |
| | Tetracaine (7) | | | | | Ethanol (1) |
| 62 | Tetracaine (10) | | Palm Oil (3) | PVA (15) | — | Water (30-70) |
| | | | | | | Ethanol (3) |
| 63 | Lidocaine (1) | Beeswax (8) | — | PVA (18) | Green Tea | Water (30-70) |
| | Prilocaine (5) | | | | Extract (5) | Ethanol (8) |
| | Tetracaine (5) | | | | | |
| 64 | Lidocaine (3) | Hyaluronic | Petrolatum | PVA (7) | — | Water (30-70) |
| | Prilocaine (2) | acid (2) | | | | Ethanol (1) |
| 65 | Lidocaine (4) | Jojoba oil | Carnauba wax | PVA (15) | — | Water (30-70) |
| | Prilocaine (2) | (4) | (10) | | | Ethanol (3) |
| 66 | Lidocaine (12) | Lecithin (6) | — | PVA (5) | Biotin | Water (30-70) |
| | Tetracaine (3) | | | | | Ethanol (10) |
| 67 | Lidocaine (10) | Cetyl | Petrolatum | PVA (12) | Biotin | Water (30-70) |
| | Tetracaine (1) | alcohol (10) | | | | Ethanol (2) |
| 68 | Lidocaine (15) | Xanthan | — | PVA (10) | Biotin | Water (30-70) |
| | Tetracaine (4) | gum (7) | | | | Ethanol (5) |
| 69 | Lidocaine (2) | Glycerin (6) | Beeswax (5) | PVA (5) | Aloe Vera | Water (30-70) |
| | Prilocaine (3) | | | | (10) | Ethanol (10) |
| | Tetracaine (10) | | | | Pantothenic | |
| | | | | | acid (10) | |
| 70 | Lidocaine (1) | Glycerin | Petrolatum + | — | Aloe Vera | Water (10) |
| | Prilocaine (1) | (10) | Mineral oil + | | (10) | |
| | | | Palm oil (50) | | Pantothenic | |
| | | | | | acid (5) | |
| 71 | Lidocaine (5) | Glycerin (1) | — | — | Aloe Vera (1) | Water (50) |
| | Prilocaine (4) | | | | Pantothenic | |
| | | | | | acid (10) | |
| 72 | Lidocaine (1) | Olive oil (3) | Paraffin Wax | — | Aloe Vera | Water (30-70) |
| | Prilocaine (5) | | (5) | | (10) | Ethanol (8) |
| | | | | | Pantothenic | |
| | | | | | acid (5) | |
| 73 | Lidocaine (3) | Shea Butter + | — | | Aloe Vera | Water (30) |
| | Prilocaine (2) | Olive oil | | | (15) | |
| | | (30) | | | Vitamin E (5) | |
| 74 | Lidocaine (4) | Glycerin (4) | — | PVA + | Aloe Vera (2) | Water (30-70) |
| | Prilocaine (2) | | | Potassium | | Ethanol (3) |
| | | | | Sorbate | | |
| | | | | (15) | | |
| 75 | Lidocaine (12) | Glycerin (6) | — | PVA (5) + | Aloe Vera | Water (30-70) |
| | Tetracaine (3) | | | Potassium | (12) | Ethanol (10) |
| | | | | Sorbate (5) | | |
| 76 | Lidocaine (10) | Beeswax | — | PVA (12) | Aloe Vera (5) | Water (30-70) |
| | | (10) | | | | Ethanol (2) |
| 77 | Lidocaine (15) | Glycerin (1) | — | PVA (10) + | Aloe Vera (7) | Water (30-70) |
| | Tetracaine (4) | | | Carbomer | | Ethanol (5) |
| | | | | (5) | | |
| 78 | Lidocaine (18) | — | Beeswax (10) | PVA (5) + | Aloe Vera | Water (30-70) |
| | Tetracaine (5) | | | Carbomer | (14) | Ethanol (8) |
| | | | | (10) | | |
| 79 | Lidocaine (22) | Glycerin (8) + | — | — | Aloe Vera | Water (30-70) |
| | Tetracaine (7) | Witch | | | (10) | Ethanol (1) |
| | | Hazel | | | Zinc Oxide | |
| | | Extract (2) | | | (20) | |
| 80 | Lidocaine (20) | Glycerin (4) | Cosmetic | — | Aloe Vera (6) | Water (30-70) |
| | Tetracaine (10) | | Wax (4) + | | | Ethanol (3) |
| | | | Beeswax (4) | | | |
| 81 | Lidocaine (1) | Beeswax (8) | Paraffin Wax | Carrageenan | Aloe Vera (5) | Water (5) |
| | Prilocaine (5) | | (60) | (18) | | |
| 82 | Lidocaine (3) | Hyaluronic | — | Cellulose | Zinc Oxide | Water (30-70) |
| | Prilocaine (2) | acid (2) | | (7) | (20) | Ethanol (1) |
| 83 | Lidocaine (4) | Jojoba oil | — | PVA (15) + | Lanolin (8) | Water (20) |
| | Prilocaine (2) | (4) | | Sodium | | |
| | | | | Benzoate | | |
| | | | | (2) | | |
| 84 | Lidocaine (12) | Lecithin (6) | — | PVA (5) + | Vitamin B, C | Water (30-70) |
| | Tetracaine (3) | | | Butylene | and E (8) | Ethanol (10) |
| | | | | Glycol (5) | | |
| 85 | Lidocaine (10) | Cetyl | — | PVA (12) | Aloe Vera | Water (30-70) |
| | Tetracaine (1) | alcohol (10) + | | | (13) | Ethanol (2) |
| | | Silicone | | | | |
| | | oil (2) | | | | |
| 86 | Lidocaine (15) | Xanthan | — | Pullulan | Aloe Vera | Water (30-70) |
| | Tetracaine (4) | gum (7) | | (10) + | (12) | Ethanol (5) |
| | | | | Cellulose | | |
| | | | | (5) | | |
| 87 | Lidocaine (1) | Petrolatum | — | Stearyl | — | Water (30-70) |
| | Prilocaine (5) | (25) | | alcohol | | Ethanol (8) |
| | | | | (18) + PVA | | |
| | | | | (10) | | |
| 88 | Lidocaine (3) | Glycerin (8) | — | Sodium | Sunflower | Water (30-70) |
| | Prilocaine (2) | | | alginate (7) | seed oil (2) + | Ethanol (1) |
| | | | | | Jojoba oil (2) | |
| 89 | Lidocaine (4) | Glycerin (4) | Cosmetic | Chitosan | — | Water (25) |
| | Prilocaine (2) | | Wax (1) | (5) | | Ethanol (3) |
| 90 | Lidocaine (12) | Glycerin (6) | Cosmetic | — | — | Water (30-70) |
| | Tetracaine (3) | | Wax (10) + | | | Ethanol (10) |
| | | | Petrolatum (5) | | | |
| 91 | Lidocaine (10) | Glycerin | Cosmetic | Algin + | — | Water (30-70) |
| | Tetracaine (1) | (10) | Wax (4) | Pectin + | | Ethanol (2) |
| | | | | Gelatin | | |
| | | | | (20) | | |
| 92 | Lidocaine (15) | Glycerin (1) | Fatty | — | — | Water (30-70) |
| | Tetracaine (4) | | alcohol(s) (3) | | | Ethanol (5) |
| 93 | Lidocaine (2) | Glycerin | Cosmetic | PVA (5) + | Aloe Vera (5) | Water (30-70) |
| | Prilocaine (3) | (10) | Wax (5) | Chitosan | Vitamin C | Ethanol (10) |
| | | | | (2) | (10) | |
| 94 | Lidocaine (1) | Squalene (2) | Cosmetic | PVA (12) | Aloe Vera (5) | Water (30-70) |
| | Prilocaine (1) | | Wax (10) | | Pantothenic | Ethanol (2) |
| | | | | | acid (1) | |
| 95 | Lidocaine (5) | Glycerin (1) | Cosmetic | PVA (10) | Aloe Vera | Water (15) |
| | Prilocaine (4) | | Wax (4) | | (15) | |
| | | | | | Pantothenic | |
| | | | | | acid (10) | |
| 96 | Lidocaine (1) | Glycerin (2) | Cosmetic | PVA (18) + | Aloe Vera | Ethanol (2) |
| | Prilocaine (5) | | Wax (10) | Potassium | (15) | |
| | | | | Sorbate (2) | Pantothenic | |
| | | | | | acid (1) | |
| 97 | Lidocaine (3) | Glycerin (8) | Candelilla | PVA (7) | Aloe Vera (8) | Water (30-70) |
| | Prilocaine (2) | | wax (10) | | Pantothenic | Ethanol (1) |
| | | | | | acid (4) | |
| 98 | Lidocaine (12) | Glycerin (6) | Cosmetic | — | Aloe Vera (3) | Water (30-70) |
| | Tetracaine (3) | | Wax (10) | | Hyaluronic | Ethanol (10) |
| | | | | | acid (2) | |
| 99 | Lidocaine (10) | Glycerin | Cosmetic | — | Pantothenic | Water (30-70) |
| | Tetracaine (1) | (10) | Wax (4) | | acid (2) + | Ethanol (2) |
| | | | | | Green Tea | |
| | | | | | extract (1) | |
| 100 | Lidocaine (15) | Xanthan | Cosmetic | — | Aloe Vera (1) | Water (30-70) |
| | Tetracaine (4) | gum (5) + | Wax (3) | | Pantothenic | Ethanol (5) |
| | | Pullulan (2) | | | acid (5) | |

Additional formulations can include any of base formulation examples 1-100, further comprising about 1-10 wt % menthol, such as about 2, 3, 4, 5, 6, 7, 8 or 9 wt % menthol.

Additional formulations can include any of base formulation examples 1-100, further comprising one or more wound-healing agents.

Additional formulations can include any of base formulation examples 1-100, further comprising a color-changing agents.

Additional formulations can include any of base formulation examples 1-100, further comprising about 1-5 wt % potassium sorbate, such as about 2, 3, or 4 wt % potassium sorbate.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that the details set forth herein do not construe a limitation to an application of the invention.

1. A composition comprising:
about 1-50 wt % of one or more local anesthetic;
about 1-30 wt % of one or more film-forming agent and/or one or more co-delivery agent; and
about 20-70 wt % water;
wherein following topical administration of the composition to tissue or skin of a patient in need thereof, one or more of the following occurs:
a) the composition changes color as the one or more local anesthetic is absorbed by the tissue or the skin;
b) the composition changes color when or after the one or more local anesthetic is completely or substantially absorbed by the tissue or the skin;
c) the composition changes color as it dries; or
d) the composition changes color when or after the composition is completely or substantially dried.

2. The composition of claim 1, wherein the composition is a self-occlusive composition.

3. The composition of claim 1, wherein the one or more local anesthetic is chosen from one or more of: lidocaine or a pharmaceutically acceptable salt thereof, tetracaine or a pharmaceutically acceptable salt thereof, prilocaine or a pharmaceutically acceptable salt thereof, benzyl alcohol, or a mixture thereof.

4. The composition of claim 1, wherein the local anesthetic is:
lidocaine or a pharmaceutically acceptable salt thereof (can be lidocaine HCL or lidocaine base);
tetracaine or a pharmaceutically acceptable salt thereof;
prilocaine or a pharmaceutically acceptable salt thereof;
benzyl alcohol;
lidocaine or a pharmaceutically acceptable salt thereof and tetracaine or a pharmaceutically acceptable salt thereof;
lidocaine or a pharmaceutically acceptable salt thereof and prilocaine or a pharmaceutically acceptable salt thereof;
lidocaine or a pharmaceutically acceptable salt thereof or a pharmaceutically acceptable salt thereof and benzyl alcohol;
tetracaine or a pharmaceutically acceptable salt thereof and prilocaine or a pharmaceutically acceptable salt thereof;
tetracaine or a pharmaceutically acceptable salt thereof and benzyl alcohol;
prilocaine or a pharmaceutically acceptable salt thereof and benzyl alcohol;
lidocaine or a pharmaceutically acceptable salt thereof, tetracaine or a pharmaceutically acceptable salt thereof and prilocaine or a pharmaceutically acceptable salt thereof; or
lidocaine or a pharmaceutically acceptable salt thereof, tetracaine or a pharmaceutically acceptable salt thereof, prilocaine or a pharmaceutically acceptable salt thereof and benzyl alcohol.

5. The composition of claim 1, further comprising about 1-20 wt % of an occlusive agent.

6. The composition of claim 1, further comprising an antimicrobial, an antiseptic, ethanol, or a mixture thereof.

7. The composition of claim 1, wherein the one or more co-delivery agent is glycerin.

8. The composition of claim 1, wherein the one or more film-forming agent is polyvinylpyrrolidone, polyvinyl alcohol, and/or potassium sorbate.

9. The composition of claim 1, further comprising one or more occlusive agent chosen from one or more of cosmetic wax, paraffin wax, or beeswax.

10. The composition of claim 1, further comprising a color-changing agent.

11. The composition of claim 1, wherein the composition changes color upon topical administration on the tissue or the skin of the patient in need thereof.

12. The composition of claim 1, wherein the composition changes color following partially complete, substantially complete, or complete, absorption of the one or more local anesthetic.

13. The composition of claim 1, further comprising one or more emulsifier, one or more emollient, one or more preservative, or a mixture thereof.

14. The composition of claim 1, further comprising one or more wound-healing agent, menthol, one or more protective agent, and/or one or more scar-reducing/preventing agent.

15. The composition of claim 1, further comprising about 1-10 wt % of one or more protective agent, pantothenic acid, panthenol, or a combination thereof.

16. The composition of claim 1, wherein the one or more co-delivery agent is chosen from one or more of the following:
Aloe vera; Beeswax; Caprylic/capric triglyceride; Cetyl alcohol; Emulsifying wax; Glycerin; Hyaluronic acid; Jojoba oil; Lecithin; Mineral oil; Olive oil; Phospholipids; Shea butter; Silicone oil; Sorbitan stearate; Squalane; Stearic acid; Sunflower seed oil; Tocopherol; Vitamin E; Urea; Vitamin C; ascorbic acid; Vitamin A; retinol; Witch hazel; Xanthan gum; Zinc oxide; or a mixture thereof.

17. The composition of claim 1, wherein the one or more film-forming agent is chosen from one or more of the following:
Acacia Senegal Gum; Acrylates Copolymer; Algin; Carbomer; Carrageenan; Cellulose; Chitosan; Ethyl cellulose; Gelatin; Hydroxyethylcellulose; Pectin; Polyvinyl Alcohol; Polyvinylpyrrolidone; Pullulan; Sodium Alginate; Sodium Carboxymethylcellulose; Xanthan Gum; Benzyl alcohol; Butylene glycol; Caprylyl glycol; Ethylhexylglycerin; Phenoxyethanol; Potassium sorbate; Sodium benzoate; Sodium dehydroacetate; Sodium metabisulfite; Tetrasodium EDTA; or a mixture thereof.

18. The composition of claim 1, further comprising one or more of the following:
alcohol; Benzalkonium chloride; Benzethonium chloride; Cetylpyridinium chloride; Chlorhexidine; Ethanol; ethyl alcohol; Hydrogen peroxide; Iodine; Isopropyl alcohol; Methylparaben; Neomycin sulfate; Povidone-iodine; Silver sulfadiazine; Tea tree oil; Melaleuca alternifolia; Triclosan; Zinc oxide; Allantoin; Aloe vera; Antioxidants; vitamin E; green tea extract; Beeswax; Biotin; Provitamin; Vitamin B7; Calendula extract; Ceramides; Chamomile extract; Cobalamin; Folic acid; folate; Glycerin; Hyaluronic acid; Jojoba oil; Lanolin; Niacinamide or Nicotinic Acid; Vitamin B3; Oat extract; Panthenol or Pantothenic; Vitamin B5: Peptides; Pyridoxine; Vitamin B6; Riboflavin; Vitamin B2; Shea butter; Sunflower seed oil; Thiamine; Vitamin B1; Titanium dioxide; Zinc oxide; or a mixture thereof.

19. The composition of claim 1, further comprising one or more occlusive agent chosen from one or more of the following:
Beeswax; Candelilla wax; Carnauba wax; Acetyl alcohol; Lanolin; Mineral oil; Ozokerite; Paraffin wax; Petrolatum; Shea butter; Squalane; Stearyl alcohol; Synthetic beeswax; Synthetic wax; or a mixture thereof.

20. The composition of claim 1, wherein the composition is biodegradable.

21. A composition comprising:
i) about 1-10 wt % or about 10-35 wt % of one or more local anesthetic;
ii) about 1-25 wt % of one or more co-delivery agent or about 1-30 wt % of one or more film-forming agent;
iii) about 20-70 wt % water;
iv) about 1-10 wt % of one or more occlusive agent and/or one or more color-changing agent.

22. The composition of claim 21, wherein the composition is a self-occlusive composition.

23. The composition of claim 21, wherein the one or more local anesthetic is lidocaine or a pharmaceutically acceptable salt thereof, prilocaine or a pharmaceutically acceptable salt thereof, tetracaine or a pharmaceutically acceptable salt thereof, benzyl alcohol, or a mixture thereof.

24. The composition of claim 21, wherein the one or more local anesthetic is:
lidocaine or a pharmaceutically acceptable salt thereof (can be lidocaine HCL or lidocaine base);
tetracaine or a pharmaceutically acceptable salt thereof;
prilocaine or a pharmaceutically acceptable salt thereof;
benzyl alcohol;
lidocaine or a pharmaceutically acceptable salt thereof and tetracaine or a pharmaceutically acceptable salt thereof;
lidocaine or a pharmaceutically acceptable salt thereof and prilocaine or a pharmaceutically acceptable salt thereof;
lidocaine or a pharmaceutically acceptable salt thereof or a pharmaceutically acceptable salt thereof and benzyl alcohol;
tetracaine or a pharmaceutically acceptable salt thereof and prilocaine or a pharmaceutically acceptable salt thereof;
tetracaine or a pharmaceutically acceptable salt thereof and benzyl alcohol;
prilocaine or a pharmaceutically acceptable salt thereof and benzyl alcohol;
lidocaine or a pharmaceutically acceptable salt thereof, tetracaine or a pharmaceutically acceptable salt thereof and prilocaine or a pharmaceutically acceptable salt thereof; or
lidocaine or a pharmaceutically acceptable salt thereof, tetracaine or a pharmaceutically acceptable salt thereof, prilocaine or a pharmaceutically acceptable salt thereof and benzyl alcohol.

25. The composition of claim 21, wherein the one or more co-delivery agent is glycerin.

26. The composition of claim 21, wherein the one or more film-forming agent is polyvinylpyrrolidone, polyvinyl alcohol, and/or potassium sorbate.

27. The composition of claim 21, wherein the one or more occlusive agent is chosen from one or more of: cosmetic wax; paraffin wax; beeswax; Candelilla wax; Carnauba wax; Acetyl alcohol; Lanolin; Mineral oil; Ozokerite; Petrolatum; Shea butter; Squalane; Stearyl alcohol; Synthetic beeswax; Synthetic wax; or a mixture thereof.

28. The composition of claim 21, wherein the composition changes color upon topical administration on tissue or skin of a patient in need thereof.

29. The composition of claim 21, wherein the composition changes color following partially complete, substantially complete, or complete, absorption of the one or more local anesthetic.

30. The composition of claim 21, further comprising one or more emulsifier, one or more emollient, one or more preservative, or a mixture thereof.
